SEQUENCE LISTING (13 sequences)

SEQ ID NO: 1 - protein, 5 aa, synthetic construct
  REGION 1..5: note = CDR sequence
HDDIS

SEQ ID NO: 2 - protein, 16 aa, synthetic construct
  REGION 1..16: note = CDR sequence
IISYAGSTYYASWAKG

SEQ ID NO: 3 - protein, 11 aa, synthetic construct
  REGION 1..11: note = CDR sequence
GVPAYSHGGDL

SEQ ID NO: 4 - protein, 13 aa, synthetic construct
  REGION 1..13: note = CDR sequence
TTSQSVYHENWLS

SEQ ID NO: 5 - protein, 7 aa, synthetic construct
  REGION 1..7: note = CDR sequence
WASTLAY

SEQ ID NO: 6 - protein, 10 aa, synthetic construct
  REGION 1..10: note = CDR sequence
AGGYGGGRYA

SEQ ID NO: 7 - protein, 119 aa, synthetic construct
  REGION 1..119: note = VH antibody domain
QVQLVESGGGLVQPGGSLRLSCAVSGIDLSHDDISWVRQAPGKGLEWVSIISYAGSTYYASWAKGRLTISKDTSKNQVVLTMTNMDPVDTATYYCARGVPAYSHGGDLWGQGTLVTVSS

SEQ ID NO: 8 - protein, 110 aa, synthetic construct
  REGION 1..110: note = VL antibody domain
DIVMTQSPATLSLSPGERATLSCTTSQSVYHENWLSWFQQKPGQPPKLLIYWASTLAYGVPSRFSGSGSGTDFTLTISSLQPEDAATYYCAGGYGGGRYAFGQGTKVEIK

SEQ ID NO: 9 - protein, 447 aa, synthetic construct
  REGION 1..447: note = Heavy chain antibody
QVQLVESGGGLVQPGGSLRLSCAVSGIDLSHDDISWVRQAPGKGLEWVSIISYAGSTYYASWAKGRLTISKDTSKNQVVLTMTNMDPVDTATYYCARGVPAYSHGGDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 10 - protein, 217 aa, synthetic construct
  REGION 1..217: note = Light chain antibody
DIVMTQSPATLSLSPGERATLSCTTSQSVYHENWLSWFQQKPGQPPKLLIYWASTLAYGVPSRFSGSGSGTDFTLTISSLQPEDAATYYCAGGYGGGRYAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 12 - protein, 242 aa, Homo sapiens
ENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 13 - protein, 266 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

(19) United States
(12) Patent Application Publication
(10) Pub. No.: US 2025/0171526 A1
(43) Pub. Date: May 29, 2025

(54) Treatment for Facioscapulohumeral Muscular Dystrophy (FSHD)

(71) Applicant: Hoffmann-La Roche Inc., Little Falls, NJ (US)

(72) Inventors: Eleni GAKI, Hitchin (GB); Tammy MCIVER, Hemingford Grey (GB); Renata SICILIANI SCALCO, Basel (CH); Kathryn Rae WAGNER, Riehen (CH); Wai Yin YEUNG, Welwyn Garden City (GB)

(73) Assignee: Hoffmann-La Roche Inc., Little Falls, NJ (US)

(21) Appl. No.: 18/902,642

(22) Filed: Sep. 30, 2024

Related U.S. Application Data
(63) Continuation of application No. PCT/EP2023/058218, filed on Mar. 30, 2023.

(30) Foreign Application Priority Data
Apr. 1, 2022 (EP) 22166261.2

(51) Int. Cl. C07K 16/22

(52) U.S. Cl. CPC C07K 16/22; C07K 2317/565; C07K 2317/76

(57) ABSTRACT

The invention provides an anti-myostatin antibody for use in the treatment, prevention, delaying progression and/or amelioration of Facioscapulohumeral muscular dystrophy (FSHD).

### CROSS REFERENCE TO RELATED APPLICATIONS

This application is a continuation of International Application No. PCT/EP2023/058218 having an International Filing Date of 30 Mar. 2023, claiming priority to application number EP 22166261.2 filed 1 Apr. 2022, each of which is incorporated herein by reference in its entirety.

### SEQUENCE LISTING

This application contains a Sequence Listing which has been submitted electronically in XML format and is hereby incorporated by reference in its entirety. Said XML copy, created on Sep. 27, 2024, is named “P37477-US_SeqList.xml” and is 17,540 bytes in size.

The invention relates to the use of an anti-myostatin antibody for the treatment of FSHD.

Facioscapulohumeral muscular dystrophy (FSHD) is a muscular dystrophy affecting roughly one in 8000 individuals making it one of the most common types of muscular dystrophy (Deenan et al. 2014). FSHD is a genetic disorder that can be caused by two different mechanisms with a common downstream pathophysiologic pathway, i.e. the derepression of the expression of the DUX4 transcription factor and resulting DUX4-mediated toxicity. FSHD type 1 (FSHD1, 95% of cases) is an autosomal dominant disorder that is due to a contraction of the D4Z4 repeats at the distal end of the subtelomeric region of chromosome 4q35. FSHD type 2 (FSHD2, 5% of cases), is a digenic disorder, due to epigenetic modification induced by mutations in genes such as SMCHD1 and DNMT3B, but presents clinically similarly to FSHD1 (Preston et al. 2020).

Symptom-onset is variable, from childhood to adulthood, but typically manifestation in the second or third decade of life. The disease is mainly characterized by skeletal muscle weakness and muscle atrophy, which affect muscles asymmetrically (Wagner 2019). Patients may present with difficulties related to facial muscle weakness such as inability to smile/whistle or to close their eyes, axial muscles weakness, scapular winging, and limb muscles weakness. The progressive nature of the disease leads to motor functional impairment, with some patients losing the ability to walk. There is considerable variability in the age of onset, severity, and rate of progression. Patients may present with a rapidly progressive infantile onset form, or a slowly progressive young-adult onset form. Respiratory involvement occurs in a subset of patients, especially those with most advanced disease (e.g., patients who are wheelchair dependent). Extramuscular findings, such as retinal vasculopathy and hearing loss, may occasionally be present and are primarily limited to those with the infantile onset form (Statland et al. 2016).

Myostatin, referred to as growth differentiation factor-8 (GDF8), is a secreted protein and is a member of the transforming growth factor-beta (TGF-beta) superfamily of proteins. Members of this superfamily possess growth-regulatory and morphogenetic properties (see, e.g., NPL1, NPL2, and PTL1). Myostatin is expressed primarily in the developing and adult skeletal muscle and functions as a negative regulator of muscle growth. Systemic overexpression of myostatin in adult mice leads to muscle wasting (see, e.g., NPL3) while, conversely, a myostatin knockout mouse is characterized by hypertrophy and hyperplasia of the skeletal muscle resulting in two- to threefold greater muscle mass than their wild type littermates (see, e.g., NPL4).

Like other members of the TGF-beta family, myostatin is synthesized as a large precursor protein containing an N-terminal propeptide domain, and a C-terminal domain considered as the active molecule (see, e.g., NPL5; PTL2). Two molecules of myostatin precursor are covalently linked via a single disulfide bond present in the C-terminal growth factor domain. Active mature myostatin (disulfide-bonded homodimer consisting of the C-terminal growth factor domain) is liberated from myostatin precursor through multiple steps of proteolytic processing. In the first step of the myostatin activation pathway, a peptide bond between the N-terminal propeptide domain and the C-terminal growth factor domain, Arg266-Asp267, is cleaved by a furin-type proprotein convertase in both chains of the homodimeric precursor. But the resulting three peptides (two propeptides and one mature myostatin (i.e., a disulfide-bonded homodimer consisting of the growth factor domains)) remain associated, forming a noncovalent inactive complex that is referred to as “latent myostatin.” Mature myostatin can then be liberated from latent myostatin through degradation of the propeptide. Members of the bone morphogenetic protein 1 (BMP1) family of metalloproteinases cleave a single peptide bond within the propeptide, Arg98-Asp99, with concomitant release of mature, active myostatin, a homodimer (see, e.g., NPL6). Moreover, the latent myostatin can be activated in vitro by dissociating the complex with either acid or heat treatment as well (see, e.g., NPL7).

Myostatin exerts its effects through a transmembrane serine/threonine kinase heterotetramer receptor family, activation of which enhances receptor transphosphorylation, leading to the stimulation of serine/threonine kinase activity. It has been shown that the myostatin pathway involves an active myostatin dimer binding to the activin receptor type IIB (ActRIIB) with high affinity, which then recruits and activates the transphosphorylation of the low affinity receptor, the activin-like kinase 4 (ALK4) or activin-like kinase 5 (ALK5). It has also been shown that the proteins Smad 2 and Smad 3 are subsequently activated and form complexes with Smad 4, which are then translocated to the nucleus for the activation of target gene transcription. It has been demonstrated that ActRIIB is able to mediate the influence of myostatin in vivo, as expression of a dominant negative form of ActRIIB in mice mimics myostatin gene knockout (see, e.g., NPL8).

A number of disorders or conditions are associated with muscle wasting (i.e., loss of or functional impairment of muscle tissue), such as muscular dystrophy (MD; including Duchenne muscular dystrophy), amyotrophic lateral sclerosis (ALS), muscle atrophy, Spinal muscular atrophy (SMA); Spinal muscular atrophy with respiratory distress type 1; Stiff person syndrome; Troyer syndrome; Guillain-Barre syndrome, organ atrophy, frailty, congestive obstructive pulmonary disease (COPD), sarcopenia, and cachexia resulting from cancer or other disorders, as well as renal disease, cardiac failure or disease, and liver disease. Patients will benefit from an increase in muscle mass and/or muscle strength; however, there are presently limited treatments available for these disorders. Thus, due to its role as a negative regulator of skeletal muscle growth, myostatin becomes a desirable target for therapeutic or prophylactic intervention for such disorders or conditions, or for monitoring the progression of such disorders or conditions. In particular, agents that inhibit the activity of myostatin may be therapeutically beneficial.

Inhibition of myostatin expression leads to both muscle hypertrophy and hyperplasia (NPL9). Myostatin negatively regulates muscle regeneration after injury and lack of myostatin in myostatin null mice results in accelerated muscle regeneration (see, e.g., NPL10). Anti-myostatin (GDF8) antibodies described in, e.g., PTL3, PTL4, PTL5, PTL6, and PTL7, and PTL8, PTL9, and PTL10 have been shown to bind to myostatin and inhibit myostatin activity in vitro and in vivo, including myostatin activity associated with the negative regulation of skeletal muscle mass. Myostatin-neutralizing antibodies increase body weight, skeletal muscle mass, and muscle size and strength in the skeletal muscle of wild type mice (see, e.g., NPL11) and the mdx mice, a model for muscular dystrophy (see, e.g., NPL12; NPL13). However, these prior art antibodies are all specific for mature myostatin but not for latent myostatin, and the strategies described for inhibiting myostatin activity have utilized antibodies that can bind to and neutralize mature myostatin. AAV-mediated follistatin, a natural myostatin antagonist, gene therapy in the tamoxifen-inducible FSHD mouse model (a disease model that recapitulates the DUX4-dependent myopathic phenotype) resulted in increased muscle mass and strength (Giesige et al. 2018).

There is no approved therapy for FSHD; thus there is a high unmet medical need as the disease can cause significant morbidity and impair the quality of life of affected patients.

The present invention provides an anti-myostatin antibody for use in the treatment, prevention, delaying progression and/or amelioration of Facioscapulohumeral muscular dystrophy (FSHD).

In an embodiment, the anti-myostatin antibody binds to latent myostatin and does not bind to mature myostatin, wherein the antibody blocks the non-proteolytic, spontaneous release of mature myostatin from latent myostatin and inhibits activation of myostatin.

In an embodiment, the anti-myostatin antibody comprises six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

In an embodiment, the anti-myostatin antibody comprises a VH domain comprising the amino acid sequence of SEQ ID NO: 7 and a VL domain comprising the amino acid sequence of SEQ ID NO: 8.

In an embodiment, the anti-myostatin antibody comprises a heavy chain comprising an amino acid sequence of SEQ ID 9 and light chain comprising an amino acid sequence of SEQ ID 10.

In an embodiment, the anti-myostatin antibody is administered every four weeks in a dose of 90 mg to an individual.

In a particular embodiment, the anti-myostatin antibody is GYM329.

### DETAILED DESCRIPTION

All publications, patent applications, patents and other references mentioned herein are incorporated by reference in their entirety.

The nomenclature used in the present application is based on IPUAC systematic nomenclature, unless indicated otherwise.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. All references cited herein, including patent applications and publications, are incorporated by reference in their entirety.

For purposes of interpreting this specification, the following definitions will apply and whenever appropriate, terms used in the singular will also include the plural and vice versa. It is to be understood that the terminology used herein is for the purpose of describing particular embodiments only, and is not intended to be limiting. Unless otherwise stated, the following terms used in the specification and claims have the meanings given below:

An “individual” or “subject”, used interchangeably, is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In a particular embodiment of the invention the subject is a human with FSHD.

The term “patient” refers to a human (such as a male or female human) who has been diagnosed with FSHD.

The term “active pharmaceutical ingredient” (or “API”) denotes the compound or molecule in a pharmaceutical composition that has a particular biological activity.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “pharmaceutical composition” refers to a preparation which is in such form as to permit the biological activity of an active ingredient contained therein to be effective, and which contains no additional components which are unacceptably toxic to a subject to which the composition would be administered. The term “pharmaceutically acceptable” denotes an attribute of a material which is useful in preparing a pharmaceutical composition that is generally safe, non-toxic, and neither biologically nor otherwise undesirable and is

The term “Cmax” (expressed in units of ng/mL) means maximum observed plasma concentration.

The term “Tmax” (expressed in units of hours, or as a median number of hours for Tmax in the study population) means the observed time to reach Cmax following drug administration; if it occurs at more than one time point Tmax is defined as the first time point with this value.

The term “AUCT0-24h” (expressed in units of ng h/mL) means the area under the plasma concentration time curve (AUC).

The term “buffer” or “buffer system” denotes a pharmaceutically acceptable excipient or excipient mixture, which stabilizes the pH of a pharmaceutical preparation. Suitable buffers are well known in the art and can be found in the literature. Particular pharmaceutically acceptable buffers comprise citric buffer, malate buffer, maleate buffer, or tartrate buffer, most particularly tartrate buffer. Particular buffer systems of the invention combinations of organic acid and selected salts thereof, e.g. tribasic sodium citrate and citric acid, malic acid and sodium malate, potassium sodium tartrate and tartaric acid, or disodium tartrate and tartaric acid, particularly potassium sodium tartrate and tartaric acid. Alternatively, the organic acid (particularly tartaric acid) can be employed alone as “acidifier” instead of the combination of acid and the corresponding salt. Independently from the buffer used, the pH can be adjusted with an acid or a base known in the art, e.g., hydrochloric acid, acetic acid, phosphoric acid, sulfuric acid and citric acid, sodium hydroxide and potassium hydroxide. Particular acidifier is tartaric acid.

The term “antioxidant” denotes pharmaceutically acceptable excipients, which prevent oxidation of the active pharmaceutical ingredient. Antioxidants comprise ascorbic acid, glutathione, cysteine, methionine, vitamin E TPGS, EDTA.

The term “therapeutically effective amount,” as used herein, refers to an amount of a compound sufficient to treat, ameliorate, or prevent the identified disease or condition, or to exhibit a detectable therapeutic, prophylactic, or inhibitory effect. The effect can be detected by, for example, an improvement in clinical condition, or reduction in symptoms. The precise effective amount for a subject will depend upon the subject's body weight, size, and health; the nature and extent of the condition; and the therapeutic or combination of therapeutics selected for administration. Where a drug has been approved by the U.S. Food and Drug Administration (FDA), a “therapeutically effective amount” refers to the dosage approved by the FDA or its counterpart foreign agency for treatment of the identified disease or condition.

As used herein, a patient “in need of GYM329 therapy” (or “in need of an anti-myostatin antibody therapy”) is a patient who would benefit from administration of GYM329.

“GYM329” also known as RO7204239 according to the present invention refers to an “anti-myostatin antibody”, wherein the antibody comprises six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6. GYM329 can also be defined by a heavy chain variable region comprising an amino acid sequence of SEQ ID 7 and light chain variable region comprising an amino acid sequence of SEQ ID 8. Methods of making and using GYM 329 are described in can be produced according to WO2016098357 and WO2017/104783. GYM 329 is known to be Fc engineered to enable remove antigen form plasma.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The terms “anti-myostatin antibody” and “an antibody that binds to myostatin” refer to an antibody that is capable of binding myostatin with sufficient affinity such that the antibody is useful as a diagnostic and/or therapeutic agent in targeting myostatin. In one embodiment, the extent of binding of an anti-myostatin antibody to an unrelated, non-myostatin protein is less than about 10% of the binding of the antibody to myostatin as measured, e.g., by a radioimmunoassay (RIA). In certain embodiments, an antibody that binds to myostatin has a dissociation constant (Kd) of 1 micro M or less, 100 nM or less, 10 nM or less, 1 nM or less, 0.1 nM or less, 0.01 nM or less, or 0.001 nM or less (e.g., 10-8 M or less, e.g., from 10-8 M to 10-13 M, e.g., from 10-9 M to 10-13 M). In certain embodiments, an anti myostatin antibody binds to an epitope of myostatin that is conserved among myostatin from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F (ab′) 2; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay, and/or conversely, the reference antibody blocks binding of the antibody to its antigen in a competition assay. An exemplary competition assay is provided herein.

A “human antibody” is one which possesses an amino acid sequence which corresponds to that of an antibody produced by a human or a human cell or derived from a nonhuman source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

The term “epitope” includes any determinant capable of being bound by an antibody. An epitope is a region of an antigen that is bound by an antibody that targets that antigen, and includes specific amino acids that directly contact the antibody. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of proteins and/or macromolecules.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The term “Fc region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, a composition comprising an antibody having an Fc region according to this invention can comprise an antibody with K447, with all K447 removed, or a mixture of antibodies with and without the K447 residue.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g., B cell receptor; BCR), etc. Such effector functions generally require the Fc region to be combined with a binding domain (e.g., an antibody variable domain) and can be assessed using various assays as disclosed, for example, in definitions herein.

The term “myostatin”, as used herein, may refer to any native myostatin from any vertebrate source, including mammals such as primates (e.g., humans) and rodents (e.g., mice and rats). Unless otherwise indicated, the term “myostatin” refers to a human myostatin protein having the amino acid sequence shown in SEQ ID NO: 11 and containing the terminal propeptide domain of human myostatin as shown in SEQ ID NO: 12 or 13. The term encompasses “full-length”, unprocessed myostatin as well as any form of myostatin that results from processing in the cell. The term also encompasses naturally occurring variants of myostatin, e.g., splice variants or allelic variants. The amino acid sequence of an exemplary human myostatin (promyostatin) is shown in SEQ ID NO: 11. The amino acid sequence of an exemplary N-terminal propeptide domain of human myostatin is shown in SEQ ID NO: 12 or 13. Active mature myostatin is a disulfide-bonded homodimer consisting of two C-terminal growth factor domains. Inactive latent myostatin is a noncovalently-associated complex of two propeptides and the mature myostatin. As disclosed herein, the antibodies of the invention bind inactive latent myostatin, but do not bind the mature active myostatin homodimer. In some embodiments, the antibodies of the invention bind an epitope within a fragment consisting of amino acids 21-100 of myostatin propeptide (SEQ ID NO: 13), but do not bind the mature active myostatin homodimer.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al., Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

A “variant Fc region” comprises an amino acid sequence which differs from that of a native sequence Fc region by virtue of at least one amino acid modification (alteration), preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include: (a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)); (b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, NIH, Bethesda, MD (1991)); (c) antigen contacts occurring at amino acid residues 27c-36 (L1), 46-55 (L2), 89-96 (L3), 30-35b (H1), 47-58 (H2), and 93-101 (H3) (MacCallum et al., J. Mol. Biol. 262:732-745 (1996)); and (d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3). Unless otherwise indicated, HVR residues and other residues in the variable domain (e.g., FR residues) are numbered herein according to Kabat et al., supra.

In some embodiments, an isolated anti-myostatin antibody of the present invention is a monoclonal antibody. In some embodiments, an isolated anti-myostatin antibody of the present invention is a human, humanized, or chimeric antibody. In some embodiments, an isolated anti-myostatin antibody of the present invention is an antibody fragment that binds to myostatin. In some embodiments, an isolated anti-myostatin antibody of the present invention is an antibody fragment that binds to latent myostatin. In some embodiments, an isolated anti-myostatin antibody of the present invention is a full length IgG antibody.

An antibody or a polypeptide comprising a variant Fc region of the invention (and optionally any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or polypeptides comprising a variant Fc region of the invention can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention will depend on the course of the disease and whether the antibody is administered for preventive or therapeutic purposes, previous therapy. The antibody or polypeptide comprising a variant Fc region of the invention is suitably administered to the patient at one time or over a series of treatments. Depending on the type and severity of the disease, about 1 micro g/kg to 15 mg/kg (e.g., 0.1 mg/kg-10 mg/kg) of antibody can be an initial candidate dosage for administration to the patient, whether, for example, by one or more separate administrations, or by continuous infusion. In particular the anti-myostatin may be administered intermittently, every week, every three weeks or particularly every four weeks, more particularly every four weeks. An initial higher loading dose, followed by one or more lower doses may be administered. The progress of this therapy is easily monitored by conventional techniques and assays.

According to the present invention, the anti myostatin maybe be formulated in a pharmaceutical formulation comprising the antibody and a pharmaceutically acceptable carrier.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

In a further aspect, the invention provides pharmaceutical formulations comprising the anti-myostatin antibody provided herein, e.g., for use in FSHD. In one embodiment, a pharmaceutical formulation comprises the anti-myostatin antibody provided herein and a pharmaceutically acceptable carrier.

The antibody or a polypeptide comprising a variant Fc region of the invention can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein. More particularly the administration according to the invention of the anti-myostatin antibody is to be administered every four weeks, more particularly via subcutaneous injections.

In a further aspect, the invention provides methods for preparing a medicament or a pharmaceutical formulation, comprising mixing the anti-myostatin antibody provided herein with a pharmaceutically acceptable carrier, e.g., for use in treating FSHD.

According to the invention, an effective amount of the myostatin inhibitor to treat a FSHD is an amount that achieves both clinical efficacy and safety. In some embodiments, the effective amount is an amount that enhances muscle function, such as force generation and motor function. In some embodiments, the effective amount is an amount that enhances motor function that requires fast-twitch fibers (e.g., type II fibers). In some embodiments, the motor function comprises eccentric contraction of a muscle. In some embodiments, an effective amount of the myostatin therapy is an amount sufficient to delay or alleviate progression of disease (e.g., muscle atrophy); maintain disease status (e.g., as measured/monitored by a suitable motor function test, plasma protein markers, metabolic markers, etc.); delay loss of a-motor neurons; prevent or delay expression of immature muscle markers; prevent, alleviate or delay intramuscular fat deposits (e.g., fatty replacement of muscle tissue); prevent metabolic dysregulation; prevent or reduce bone loss or frequency of bone fracture; increase an Expanded Hammersmith Functional Motor Scale score by >1 point 1 as compared to control that does not receive the myostatin inhibitor; slow the rate of deterioration; delay regression (e.g., progressive decrease) of an Expanded Hammersmith Functional Motor Scale over a period of 12 months, 24 months or 36 months; and/or, increase a CHOP INTEND score by >1 point as compared to control that does not receive the myostatin inhibitor; and/or, increase a MFM-32 score by >1 point as compared to control that does not receive the myostatin inhibitor.

According to the here within described invention more particular embodiments of the invention are described below:

Embodiment 1: A method for the treatment, prevention, delaying progression and/or amelioration of FSHD in a subject in need thereof, wherein an anti-myostatin antibody is administered to the subject in need thereof.

Embodiment 2: The method of embodiment 1, wherein the anti-myostatin antibody binds to latent myostatin and does not bind to mature myostatin, wherein the antibody blocks the non-proteolytic, spontaneous release of mature myostatin from latent myostatin and inhibits activation of myostatin.

Embodiment 3: The method of embodiment 1 or 2, wherein the anti-myostatin antibody comprises the six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 4: The method of embodiments 1 to 3, wherein the anti-myostatin antibody comprises a VH chain comprising the amino acid sequence of SEQ ID 7 and a VL chain comprising the amino acid sequence of SEQ ID 8.

Embodiment 5: The method of embodiments 1 to 4, wherein the anti-myostatin antibody comprises a heavy chain comprising the amino acid sequence of SEQ ID 9 and a light chain comprising the amino acid sequence of SEQ. ID. 10.

Embodiment 6. The method of embodiments 1 to 5, wherein the anti-myostatin antibody is administered every four weeks in a dose of 90 mg to a subject, preferably a human subject.

Embodiment 7: The method of embodiments 1 to 6, wherein the anti-myostatin antibody is administered subcutaneously.

Embodiment 8: A use of an anti-myostatin antibody for the manufacture of a medicament for the treatment, prevention, delaying progression and/or amelioration of FSHD in a subject in need thereof.

Embodiment 9: The use of embodiment 8, wherein the anti-myostatin antibody binds to latent myostatin and does not bind to mature myostatin, wherein the antibody blocks the non-proteolytic, spontaneous release of mature myostatin from latent myostatin and inhibits activation of myostatin.

Embodiment 10: The use of embodiment 8 or 9, wherein the anti-myostatin antibody comprises the six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 11: The use of embodiments 8 to 10, wherein the anti-myostatin antibody comprises a VH chain comprising the amino acid sequence of SEQ ID 7 and a VL chain comprising the amino acid sequence of SEQ ID 8.

Embodiment 12: The use of embodiments 8 to 11, wherein the anti-myostatin antibody comprises a heavy chain comprising the amino acid sequence of SEQ ID 9 and a light chain comprising the amino acid sequence of SEQ. ID. 10.

Embodiment 13. The use of embodiments 8 to 12, wherein the anti-myostatin antibody is administered every four weeks in a dose of 90 mg to a subject, preferably a human subject.

Embodiment 14: The use of embodiments 8 to 13, wherein the anti-myostatin antibody is administered subcutaneously.

Embodiment 15: A pharmaceutical formulation for use in the treatment of FSHD comprising an anti-myostatin antibody.

Embodiment 16: The pharmaceutical formulation of embodiment 15, wherein the anti-myostatin antibody binds to latent myostatin and does not bind to mature myostatin, wherein the antibody blocks the non-proteolytic, spontaneous release of mature myostatin from latent myostatin and inhibits activation of myostatin.

Embodiment 17: The pharmaceutical formulation of embodiment 15 or 16, wherein the anti-myostatin antibody comprises the six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 18: The pharmaceutical formulation of embodiments 15 to 17, wherein the anti-myostatin antibody comprises a VH chain comprising the amino acid sequence of SEQ ID 7 and a VL chain comprising the amino acid sequence of SEQ ID 8.

Embodiment 19: The pharmaceutical formulation of embodiments 15 to 18, wherein the anti-myostatin antibody comprises a heavy chain comprising the amino acid sequence of SEQ ID 9 and a light chain comprising the amino acid sequence of SEQ. ID. 10.

Embodiment 20: The pharmaceutical formulation of embodiments 15 to 19, wherein the anti-myostatin antibody comprises a heavy chain comprising the amino acid sequence of SEQ ID 9 and a light chain comprising the amino acid sequence of SEQ. ID. 10.

Embodiment 21: The pharmaceutical formulation of embodiments 15 to 20, wherein the pharmaceutical formulation is administered every four weeks in a dose of 90 mg to a subject, preferably a human subject.

Embodiment 22: The pharmaceutical formulation of embodiments 15 to 21 wherein the pharmaceutical formulation is administered subcutaneously.

Embodiment 23: A medicament for the treatment of FSHD comprising an anti-myostatin antibody.

Embodiment 24: The medicament of embodiment 23, wherein the anti-myostatin antibody binds to latent myostatin and does not bind to mature myostatin, wherein the antibody blocks the non-proteolytic, spontaneous release of mature myostatin from latent myostatin and inhibits activation of myostatin.

Embodiment 25: The medicament of embodiment 23 or 24, wherein the anti-myostatin antibody comprises the six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 26: The medicament of embodiments 23 to 25, wherein the anti-myostatin antibody comprises a VH chain comprising the amino acid sequence of SEQ ID 7 and a VL chain comprising the amino acid sequence of SEQ ID 8.

Embodiment 27: The medicament of embodiments 23 to 26, wherein the anti-myostatin antibody comprises a heavy chain comprising the amino acid sequence of SEQ ID 9 and a light chain comprising the amino acid sequence of SEQ. ID. 10.

Embodiment 28: The medicament of embodiments 23 to 27, wherein the anti-myostatin antibody comprises a heavy chain comprising the amino acid sequence of SEQ ID 9 and a light chain comprising the amino acid sequence of SEQ. ID. 10.

Embodiment 29: The medicament of embodiments 23 to 28, wherein the medicament is administered every four weeks in a dose of 90 mg to a subject, preferably a human subject.

Embodiment 30: The medicament of embodiments 23 to 29, wherein the medicament is administered subcutaneously.

TABLE 1
Anti-Myostatin Antibody GYM329 CDR sequences

| CDR | Antibody GYM329 | SEQ ID # |
|---|---|---|
| CDRH1 | HDDIS | SEQ ID 1 |
| CDRH2 | IISYAGSTYYASWAKG | SEQ ID 2 |
| CDRH3 | GVPAYSHGGDL | SEQ ID 3 |
| CDRL1 | TTSQSVYHENWLS | SEQ ID 4 |
| CDRL2 | WASTLAY | SEQ ID 5 |
| CDRL3 | AGGYGGGRYA | SEQ ID 6 |

TABLE 2
Anti-Myostatin Antibody GYM329
amino acid sequences

| | Amino Acid Sequence | SEQ ID # |
|---|---|---|
| Heavy | QVQLVESGGGLVQPGGSLRLSCA | SEQ ID 7 |
| Chain | VSGIDLSHDDISWVRQAPGKGLE | |
| Variable | WVSIISYAGSTYYASWAKGRLTIS | |
| Region | KDTSKNQVVLTMTNMDPVDTAT | |
| | YYCARGVPAYSHGGDLWGQGTL | |
| | VTVSS | |
| Light Chain | DIVMTQSPATLSLSPGERATLSCT | SEQ ID 8 |
| variable | TSQSVYHENWLSWFQQKPGQPPK | |
| Region | LLIYWASTLAYGVPSRFSGSGSGT | |
| | DFTLTISSLQPEDAATYYCAGGYG | |
| | GGRYAFGQGTKVEIK | |
| Heavy | QVQLVESGGGLVQPGGSLRLSCA | SEQ ID 9 |
| Chain | VSGIDLSHDDISWVRQAPGKGLE | |
| | WVSIISYAGSTYYASWAKGRLTIS | |
| | KDTSKNQVVLTMTNMDPVDTAT | |
| | YYCARGVPAYSHGGDLWGQGTL | |
| | VTVSSASTKGPSVFPLAPSSKSTSG | |
| | GTAALGCLVKDYFPEPVTVSWNS | |
| | GALTSGVHTFPAVLQSSGLYSLSS | |
| | VVTVPSSSLGTQTYICNVNHKPSN | |
| | TKVDKKVEPKSCDKTHTCPPCPAP | |
| | EYLGGDSVFLFPPKPKDVLMISRT | |
| | PEVTCVVIDVSHEDPEVKFNWYV | |
| | DGVEVHNAKTKPREEQYNSTYRV | |
| | VSVLPVLHRDWLNGKEYKCKVS | |
| | NKALPKPIEKTISKAKGQRREPQV | |
| | YTLPPSREEMTKNQVSLTCLVKGF | |
| | YPSDIAVEWESNGQPENNYKTTPP | |
| | VLDSDGSFFLYSKLTVDKSRWQQ | |
| | GNVFSCSVLHEALHAHTTRKELSL | |
| | SP | |
| Light Chain | DIVMTQSPATLSLSPGERATLSCT | SEQ 10 |
| | TSQSVYHENWLSWFQQKPGQPPK | |
| | LLIYWASTLAYGVPSRFSGSGSGT | |
| | DFTLTISSLQPEDAATYYCAGGYG | |
| | GGRYAFGQGTKVEIKRTVAAPSV | |
| | FIFPPSDEQLKSGTASVVCLLNNF | |
| | YPREAKVQWKVDNALQSGNSQE | |
| | SVTEQDSKDSTYSLSSTLTLSKAD | |
| | YEKHKVYACEVTHQGLSSPVTKS | |
| | FNRGEC | |

The following examples are intended merely to illustrate the practice of the present invention and is not provided by way of limitation.

### Example 1

Phase II, multi-center, randomized, double-blind, placebo-controlled study to evaluate the pharmacodynamics, safety, tolerability, pharmacokinetics, and efficacy of GYM329 (RO7204239) in ambulant adult participants with FSHD.

The study will enroll approximately 48 participants. There is a screening period (up to 28 days) to determine eligibility of a participant, followed by enrollment into the study, if all the requirements are met. Participants will then complete a 3-week pre-treatment period to collect baseline movement data via wearable device on the wrist and the ankle before randomization (1:1, RO7204239: placebo) for the 52-week double-blind treatment period.

RO7204239 or placebo will be administered every 4 weeks by SC injection into the abdomen.

Once participants complete the 52-week double blind treatment period, participants will have the option to roll over into the OLE period where all participants will receive RO7204239 for an additional 52 weeks, unless the development of RO7204239 in FSHD is stopped.

The primary analysis will occur after 52 weeks of treatment, as measured by magnetic resonance imaging (MRI) and exploratory endpoints of muscle strength and motor function.

The nature, frequency, severity, and timing of adverse events, serious adverse events, local and systemic injection reactions, vital signs, laboratory parameters, ECGs, and echocardiogram tests will be assessed on a regular basis by an unblinded iDMC.

Blood samples for the assessment of PK, PD, and ADA profile will be obtained from all participants.

An Independent Review Facility will collect, store, and review imaging data. Individuals who do not meet the criteria for participation in this study (screen failure) may qualify for one re-screening opportunity (for a total of 2 screenings per individual) at the investigator's discretion. Individuals are not required to re-sign the consent form if they are re-screened within 29 days after previously signing the consent form. The investigator will record reasons for screen failure in the screening log.

The duration of the study for each participant is divided as follows:
Screening: Day −52 to Day −24
Enrollment: Day −23
Pretreatment period: Day −22 to Day −2
Randomization: Day −1
Baseline: Day −1 to Day 1

Baseline assessments will be completed prior to study treatment dosing.
Start of treatment: Day 1
Double-blind treatment period: 52 weeks
Open-label extension period: 52 weeks
Safety Follow-Up: 3 months after final dose of RO7204239

### Rationale for Study Design

### Rationale for Study Population

This study will enroll individuals genetically diagnosed with FSHD1 or FSHD2, as both FSHD types have the same phenotype (Hamel and Tawil 2018). RO7204239 has the potential to be effective in both patients with FSHD1 and patients with FSHD2, given that its mechanism of action is not dependent on the underlying FSHD genetics.

Only participants who are ambulant will be included in the study. Data from patients with neuromuscular diseases have indicated that circulating concentrations of myostatin are decreased with disease progression (Burch et al. 2017). Considering that myostatin is the target of RO7204239, ambulant FSHD patients are considered to have the greatest potential to demonstrate the benefit of an anti-myostatin therapy in this study due to greater functional muscle preservation as a result of a less advanced disease.

### Rationale for Control Group

The control group in this study receives placebo.

FSHD is generally a slowly progressive disease but highly variable in presentation and rate of progression. There are limited natural history data in FSHD in terms of expected changes in skeletal muscle MRI parameters and clinical outcomes within one year; thus it is important to differentiate these changes from any effects due to RO7204239 treatment.

As there are no approved treatments for FSHD, the use of a placebo control is considered ethical and justified. Moreover, symptomatic medications (e.g. for pain) that may be part of the standard of care treatment of patients with FSHD are allowed as concomitant medications in this study.

All participants enrolled in this study will ultimately receive RO7204239 treatment. Participants receiving placebo will have the option to receive RO7204239 in the OLE period for 52 weeks.

Given all the above, the use of placebo control is justified and necessary for a robust assessment of the pharmacodynamics, safety, tolerability, and efficacy of RO7204239.

### Rationale for Primary Endpoint Selection

The primary endpoint is based on the MRI assessment of change in skeletal muscle volume. Quantitative muscle MRI has been shown to have a strong correlation with clinical outcome measures in FSHD and able to detect early muscle changes (Mul et al. 2017). An increase in contractile muscle volume is considered an evidence of the bioactivity of RO7204239, based on its mechanism of action.

Given that fat infiltration can be heterogeneous along the muscles' length (Janssen et al. 2014), MRI volumetric measurement was selected for the assessment of the primary endpoint compared to measurements of cross-sectional area, which are considered surrogates of changes in muscle volume and will be assessed as secondary endpoints.

The quadriceps muscle was selected for the assessment of the primary endpoint as it is a muscle that is affected in FSHD (Tasca et al. 2016), with variable involvement and degree of fatty infiltration (Mul et al, 2017) allowing for an effect to be observed. In addition, it is a functionally important muscle for walking and ascending/descending stairs, and its contractile cross sectional area correlates significantly with muscle strength in FSHD (Lassche et al. 2020).

### Justification for Dose and Schedule

RO7204239 is a humanized mAb administered SC every 4 weeks into the abdomen. In Study BP40484, a SAD study investigating the pharmacodynamics, safety, tolerability, pharmacokinetics, and immunogenicity of RO7204239 in healthy adult participants, single doses up to 90 mg were well tolerated and showed target engagement (i.e., sustained total latent, free latent, and mature myostatin suppression).

Therefore, a dose of 90 mg every 4 weeks was chosen as the dosing regimen for this study. This is predicted to provide ≥95% inhibition of total latent myostatin, and the complete myostatin inhibition is expected to translate into the maximum possible effect in this study.

### Study Population

Approximately 48 participants with FSHD will be enrolled in this study.

### Inclusion Criteria

Participants are eligible to be included in the study only if all of the following criteria apply:
Age ≥18 years and ≤65 years at the time of signing the Informed Consent Form
Genetic confirmation of FSHD1 or FSHD2, including one of the following:
   For FSHD1: a heterozygous pathogenic contraction of the D4Z4 repeat array in the subtelomeric region of chromosome 4q35 on the permissive chromosome 4 haplotype
   For FSHD2: hypomethylation of the D4Z4 repeat array in the subtelomeric region of chromosome 4q35 on the permissive chromosome 4 haplotype, and a heterozygous SMCHD1 pathogenic variant or a heterozygous DNMT3B pathogenic variant
Clinical findings consistent with FSHD as per investigator's clinical judgement
Ambulant, where ambulant is defined as able to walk/run unassisted (i.e., without the use of assisted devices such as canes, crutches or walkers, or person/hand-held assistance) 10 meters in >4 and ≤12 seconds at screening
Ricci Clinical Severity Scale score ≥2.5 and ≤4
Agreement to maintain the same frequency and intensity of physiotherapy, occupational therapy and other forms of exercise therapy during the clinical study.
Able and willing to comply with the study protocol and to complete all study procedures, measurements, and visits
For female participants of childbearing potential: agreement to remain abstinent (refrain from heterosexual intercourse) or use contraception, as defined below:
   Female participants must remain abstinent or use contraceptive methods with a failure rate of <1% per year during the treatment period and for 17 months after the final dose of RO7204239.
   A female participant is considered to be of childbearing potential if she is postmenarchal, has not reached a postmenopausal state (≥12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis). Per this definition, a female participant with tubal ligation is considered to be of childbearing potential. The definition of childbearing potential may be adapted for alignment with local guidelines or regulations.
   Examples of contraceptive methods with a failure rate of <1% per year include bilateral tubal ligation, male sterilization, hormonal contraceptives that inhibit ovulation, hormone-releasing intrauterine devices, and copper intrauterine devices.
   The reliability of sexual abstinence should be evaluated in relation to the duration of the clinical trial and the preferred and usual lifestyle of the individual. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence will be described in the local Informed Consent Form.
For male participants: agreement to remain abstinent (refrain from heterosexual intercourse) or use contraceptive methods, and agree to refrain from donating sperm, as defined below:
   With a female partner of childbearing potential who is not pregnant, male participants must remain abstinent or use a condom plus an additional contraceptive method that together result in a failure rate of <1% per year during the treatment period and for 120 days after the final dose of RO7204239. Male participants must refrain from donating sperm during this same period.
   With a pregnant female partner, male participants must remain abstinent or use a condom during the treatment period and for 120 days after the final dose of RO7204239 to avoid exposing the embryo.
   The reliability of sexual abstinence should be evaluated in relation to the duration of the clinical trial and the preferred and usual lifestyle of the individual. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence will be described in the local Informed Consent Form.

### Exclusion Criteria

Participants are excluded from the study if any of the following criteria apply:
Pregnancy or breastfeeding, or intention of becoming pregnant during the study or within 17 months after the final dose of RO7204239.
   Female participants of childbearing potential must have a negative serum pregnancy test result within 14 days prior to initiation of study treatment.
Current or previous administration of anti-myostatin therapies
Treatment with any investigational therapy within 90 days prior to screening, or 5 half-lives of the drug, whichever is longer
Contraindications for MRI scans (including, but not restricted to, claustrophobia, pacemaker, artificial heart valves, cochlear implants, presence of foreign metal objects in heart or body, including intracranial vascular clips, insulin pumps, etc.), difficulties maintaining a prolonged supine position, or any other clinical history or examination finding that would pose a potential hazard in combination with MRI
Presence of clinically significant ECG abnormalities from average of triplicate measurement at screening or cardiovascular disease (e.g., cardiac insufficiency, coronary artery disease, cardiomyopathy, congestive heart failure, family history of congenital long QT syndrome, family history of sudden death) indicating a safety risk for participants
Presence of clinically significant abnormal findings in echocardiography at screening, with the exception of mitral valve prolapse which does not exclude participants from the study
Any major illness within 1 month before screening
Ascertained or presumptive hypersensitivity (e.g., anaphylactic reaction) to RO7204239, or to the constituents of its formulation
Concomitant disease or a medical condition or abnormality in clinical laboratory tests that could interfere with, or treatment of which might interfere with, the conduct of the study, or that would pose an unacceptable risk to the participant in this study. In the case of uncertain or questionable results, tests performed during screening may be repeated before randomization to confirm eligibility
History of malignancy (except in situ basal cell carcinoma of the skin, and in situ carcinoma of the cervix of the uterus that have been excised and resolved with documented clean margins on pathology)
Any clinically relevant history of anaphylactic reaction requiring inotropic support
Any abnormal skin conditions, pigmentation or lesions in the area intended for SC injection (abdomen) and that would prevent visualization of potential injection site reactions to RO7204239.
Immobilization, surgical procedures, fracture, or trauma to the upper or lower limbs within 90 days prior to screening or longer, if judged by the investigator that it may affect motor function assessment
Planned to have any surgery which may affect motor function assessment during the study including participants who have had surgery of scapular fixation in the 12 months preceding screening or planned until the end of the study.
Substance abuse within 12 months prior to screening or are at risk of substance abuse per investigator's judgement
Use of the following medications within 90 days prior to enrollment:
   Salbutamol or another β2-adrenergic agonist taken orally
   Creatine
   Growth hormone
   IGF-1
   Testosterone, Oxandrolone or other anabolic steroid
   Chronic oral or parenteral use of corticosteroids (inhaled corticosteroid use is allowed) unless required to manage injection reactions
   Agents anticipated to increase or decrease muscle volume or strength

### Study Treatment(s) and Concomitant Therapy

Study treatment is defined as any investigational treatment, marketed product, placebo, or medical device intended to be administered to a study participant according to the study protocol.

The investigational medicinal products (IMP) for this study are RO7204239 and placebo.

### Study Treatments Administered

Table 1 provides a description of assigned study treatments for this study.

TABLE 1
Study Treatment Description

| | RO7204239 | RO7204239-matching Placebo |
|---|---|---|
| Use | Experimental | Placebo comparator |
| Type of medicinal | IMP | IMP |
| product | | |
| Drug form | 1.0 mL of sterile, preservative- | 1.0 mL of sterile, preservative- |
| | free solution for injection | free solution for injection |
| | containing 80 mg of RO7204239 | |
| Unit Dose Strength(s) | 80 mg/mL | NA |
| Dosage Level | 90 mg Q4W | NA |
| Formulation(s) | Refer to Investigator's Brochure | NA |
| Packaging | 3-mL glass vials | 3-mL glass vials |
| Labeling | Per local requirements | Per local requirements |
| Route of administration | SC injection | SC injection |
| Source | Sponsor ^{a} | Sponsor ^{a} |

IMP = investigational medicinal product; NA = not applicable; Q4W = every 4 weeks; SC = subcutaneous.
^{a} Diluent will also be supplied by the Sponsor.

### RO7204239 (GYM329)

RO7204239 will be provided in 3-mL glass vials containing 80 mg/mL and must be prepared for dosing under appropriate aseptic conditions. The solution must be diluted as necessary and filtered prior to injection using a needle filter. The solution ready for injection should preferably be used immediately. Detailed instructions are provided in the Pharmacy Manual.

RO7204239 will be administered every 4 weeks by SC injection in the abdomen. The administration volume is described in the Pharmacy Manual. Each injection should be administered in a separate location in rotating quadrants of the abdomen at each study visit where this treatment is administered. RO7204239 will be administered at the clinical site by site staff. Participants will be monitored at the site for a minimum of 6 hours after the first two administrations of the double-blind treatment period and the first two administrations of the OLE. For all other administrations, participants will be monitored for 2 hours (or longer if deemed necessary by the investigator/site staff).

Only participants enrolled in the study may receive RO7204239, only authorized staff may supply RO7204239, and only authorized staff or trained study personnel may administer the study drug.

### Placebo

Placebo of identical appearance, composition (except RO7204239) and identical volume to RO7204239 will be administered by SC injection to all participants randomized to placebo, and will be administered in the same dose regimen (every 4 weeks).

### Efficacy Assessments

### FSHD-Composite Functional Outcome Measure

The FSHD-Composite Functional Outcome Measure (FSHD-COM) is an assessment of disease-relevant areas of functional burden. It is a performance-based functional composite outcome measure which combines multiple functional domains and individual evaluator-administered items into one measure and captures key components of patient-identified disease burden. Lower scores correlate with better function (Eichinger et al. 2018).

The FSHD-COM includes 18 items grouped into 5 body regions (leg function, arm and shoulder function, trunk function, hand function, and balance). Each item is scored on a 5-point ordinal scale, with 0 representing “unaffected/normal performance” and 4 representing “severely affected”. A total score will be calculated based on the sum of the item scores and will range from 0-72, with a higher score representing a higher functional burden. Subscale scores will also be calculated for each of the 5 body regions.

The scale will be administered by a trained Clinical Evaluator (Physical Therapist or other suitably qualified professional who has received training on the administration of the FSHD-COM). If possible, the same assessor should follow the participant throughout the study. Scores will be recorded on the scoring sheet and on the eCRF.

The FSHD-COM takes about 35 minutes to complete, including a 10 minute rest prior to the six minute walk distance.

### Muscle Strength by Myometry

Muscle strength will be assessed using handheld dynamometry. All assessments should be done on the right and left side.

The following will be tested:
elbow flexion
elbow extension
shoulder abduction
knew flexion
knee extension
ankle dorsiflexion

For each testing, three values of maximum muscular force (peak force) will be collected on the right and left side at timepoints specified in the Schedule of Assessments. All values will be transferred to the appropriate eCRF. For the purpose of data analysis, only the highest value per testing per side per visit will be analyzed.

Training of clinical evaluators and quality assurance of site myometry assessment administration will be described in the Motor Function and Strength Assessments study manual and site manual. Video recording of the myometry assessments at site may be performed to allow for central Quality Review by expert physiotherapists. All collected videos will be masked (anonymized) using blurring technology prior to being subjected to Central

### Primary Endpoint

The primary efficacy endpoint is the percentage change from baseline in CMV of quadriceps muscle at Week 52 of treatment, as defined in Section 3 (Table 3). The percentage change from baseline in the CMV of quadriceps muscles as assessed by MRI will be derived at each timepoint.

The primary estimand is defined as the following attributes:

Population: Ambulant at the time of randomization of FSHD1 or FSHD2 participants as defined by study inclusion and exclusion criteria, aged 18-65 years at the time of signing the Informed Consent Form
Variable: CMV of quadriceps muscle as assessed by MRI
Treatment:
   RO7204239 90 mg every 4 weeks by SC injection or
   Placebo every 4 weeks by SC injection
Intercurrent events:
   Early withdrawal from study treatment (RO7204239 or placebo)
   Death
Handling of intercurrent events:
   Early withdrawal from study treatment for study drug-related reasons (e.g. treatment-related adverse events, lack of efficacy, etc.). All available data will be included in the analysis for treatment-policy strategy.
   Early withdrawal from study treatment for non-study drug-related reasons (e.g. Death). A hypothetical treatment strategy will be applied assuming participants continue their randomized treatment until the primary analysis timepoint.
Population-level summary: The difference between the RO7204239 arm and the placebo arm in the mean percentage change from baseline in the CMV of quadriceps muscle as assessed by MRI after 52 weeks of treatment.

The percentage change from baseline in the CMV at week 52 is defined as

$$\frac{CMV at Week 52 - CMV at baseline}{CMV at baseline} \times 100 \%$$

The hypothesis to be tested is the difference in the mean percentage change from baseline in the CMV of quadriceps muscle as assessed by MRI at Week 52 between the RO7204239 arm and the placebo arm (δ):
H₀:δ=0 (null) versus H₁:δ=0 (alternative)

Testing will be performed at a two-sided 5% significance level:

The estimate of the treatment effect will be computed using a Mixed Model Repeated Measures (MMRM) analysis. The model will include the percentage change from baseline in the CMV of quadriceps muscle as assessed by MRI as the dependent variable. Independent variables of the model will include baseline CMV, treatment group, time, and treatment-by-time interaction. An unstructured variance-covariance matrix structure will be applied. The estimated treatment difference in the mean percentage change from baseline in the CMV of quadriceps muscle as assessed by MRI at Week 52 between RO7204239 and placebo will be presented with the 95% confidence interval. The percentage change from baseline in the CMV, the actual CMV, and the actual change from baseline in the CMV will also be summarized at each timepoint by treatment arm and by study period.

Efficacy results from patients from the enrolled population but not in the efficacy analysis population will be listed separately, if applicable.

The primary safety endpoints are as follows, as defined in Section 3 Table 2.

Incidence, severity, and causal relationship of adverse events, with severity determined according to NCI CTCAE v5.0

Change from baseline in vital signs, physical findings, ECG, echocardiogram, and clinical laboratory results
Incidence of local and systemic injection reactions
Incidence of abnormal laboratory findings
Incidence of abnormal ECG parameters
Incidence of abnormal echocardiographic parameters
Incidence of abnormal vital signs

All safety analyses will be mainly based on the safety population.

Safety will be assessed through summaries of exposure to study treatment, adverse events, abnormal results in vital signs, ECG, echocardiographic, and laboratory assessments, and may also be assessed through summaries results on the changes in physical findings, echocardiogram, laboratory, vital signs, and ECG test results.

Study treatment exposure (such as treatment duration, total dose received, and dose modifications) will be summarized with descriptive statistics by treatment arm and by study period.

All verbatim adverse event terms will be mapped to Medical Dictionary for Regulatory Activities thesaurus terms, and adverse event severity will be graded according to NCI CTCAE v5.0. All adverse events, serious adverse events, adverse events leading to death, adverse events of special interest, and adverse events leading to study treatment discontinuation that occur on or after the first dose of study treatment (i.e., treatment-emergent adverse events) will be summarized by mapped term, appropriate thesaurus level, and severity grade. For events of varying severity, the highest grade will be used in the summaries. The adverse event results will be summarized by treatment arm and by study period. Deaths and cause of death will be listed. Any serious adverse events observed during the pre-treatment period will also be summarized and listed for the enrolled population.

Abnormal results from laboratory, vitals, echocardiographic, and ECG test assessments will be summarized at each timepoint by treatment arm and by study period. Relevant change from baseline results in laboratory, vital sign (pulse rate, respiratory rate, blood pressure, pulse oximetry, and temperature), echocardiographic, and ECG data may be displayed by time, with Grades identified where appropriate. Additionally, a shift table of selected laboratory tests may be used to summarize the baseline and maximum post-baseline severity grade where appropriate.

### Secondary Endpoints

The secondary efficacy endpoints are as follows, as defined in Sectio3 (Table 3):
Change from baseline in serum concentrations of total and free latent myostatin, and mature myostatin
Percentage change from baseline in CMV of quadriceps muscle as assessed by MRI at Week 28 of treatment
Percentage change from baseline in CMV of tibialis anterior muscle as assessed by MRI at Week 28 and 52 of treatment
Percentage change from baseline in CMV of biceps brachii muscle as assessed by MRI at Week 28 and 52 of treatment
Percentage change from baseline in the contractile area of skeletal muscle in the proximal lower limb muscles as assessed by MRI at Week 28 and 52 of treatment
Percentage change from baseline in the contractile area of skeletal muscle in the distal lower limb muscles as assessed by MRI at Week 28 and 52 of treatment
Percentage change from baseline in the contractile area of skeletal muscles in the proximal upper limb as assessed by MRI at Week 28 and 52 of treatment
Change from baseline in the fat fraction of proximal lower limb muscles as assessed by MRI at Week 28 and 52 of treatment
Change from baseline in the fat fraction of distal lower limb muscles as assessed by MRI at Week 28 and 52 of treatment
Change from baseline in the fat fraction of proximal upper limb muscles as assessed by MRI at Week 28 and 52 of treatment

All analysis of the secondary efficacy endpoint will be performed on data up to Week 52 for each individual in the efficacy analysis population. Efficacy results from patients included in the enrolled population but not in the efficacy analysis population will be listed separately, if applicable.

Each of the secondary endpoints will be tested at a two-sided 5% significance level without controlling multiplicity.

The continuous secondary efficacy endpoints will be analyzed using the MMRM similarly as described for the primary efficacy endpoints. The estimated treatment difference and corresponding 95% confidence interval will be reported.

All secondary efficacy endpoint results will also be summarized at each timepoint by treatment arm and by study period.

TABLE 3

| Primary Objectives | Corresponding Endpoint |
|---|---|
| Evaluation of pharmacodynamic | Percentage change from baseline in contractile |
| effects (MRI) of RO7204239 | muscle volume (CMV) of quadriceps muscles as |
| compared with placebo | assessed by MRI bilaterally at Week 52 of treat- |
| | ment |
| Evaluation of the safety of | Incidence, severity, and causal relationship of |
| RO7204239 compared with | adverse events, with severity determined according |
| placebo | to NCI CTCAE v5.0 |
| | Change from baseline in vital signs, physical |
| | findings, ECG, echocardiogram, and clinical |
| | laboratory results |
| | Incidence of local and systemic injection reactions |
| | Incidence of abnormal laboratory findings |
| | Incidence of abnormal ECG parameters |
| | Incidence of abnormal echocardiographic |
| | parameters |
| | Incidence of abnormal vital signs |
| Secondary Objectives | Corresponding Endpoints |
| Evaluation of pharmacodynamic | Change from baseline in serum concentrations of |
| effects (serum and MRI) of | total and free latent myostatin, and mature |
| RO7204239 compared to placebo | myostatin |
| | Percentage change from baseline in CMV of |
| | quadriceps muscles as assessed by MRI bilaterally |
| | at Week 28 of treatment |
| | Percentage change from baseline in CMV of |
| | tibialis anterior muscles as assessed by MRI |
| | bilaterally at Weeks 28 and 52 of treatment |
| | Percentage change from baseline in CMV of |
| | biceps brachii muscles as assessed by MRI |
| | bilaterally at Weeks 28 and 52 of treatment |
| | Percentage change from baseline in the contractile |
| | area of skeletal muscle in the proximal lower limb |
| | muscles as assessed by MRI bilaterally at Weeks |
| | 28 and 52 of treatment |
| | Percentage change from baseline in the contractile |
| | area of skeletal muscle in the distal lower limb |
| | muscles as assessed by MRI bilaterally at Weeks |
| | 28 and 52 of treatment |
| | Percentage change from baseline in the contractile |
| | area of skeletal muscles in the proximal upper |
| | limb muscles as assessed by MRI bilaterally at |
| | Weeks 28 and 52 of treatment |
| | Change from baseline in the fat fraction of |
| | proximal lower limb muscles as assessed by MRI |
| | bilaterally at Weeks 28 and 52 of treatment |
| | Change from baseline in the fat fraction of distal |
| | lower limb muscles as assessed by MRI bilaterally |
| | at Weeks 28 and 52 of treatment |
| | Change from baseline in the fat fraction of |
| | proximal upper limb muscles as assessed by MRI |
| | bilaterally at Weeks 28 and 52 of treatment |
| Evaluation of PK parameters for | Serum concentration of RO7204239 at specified |
| RO7204239 | timepoints |
| | Cₘₐₓ |
| | AUC |
| | C_{trough} |
| | Other PK parameters as appropriate |
| Evaluation of the immune | Prevalence of ADAs at baseline and incidence |
| response to RO7204239 | of ADAs during the study |
| Exploratory Objectives | Corresponding Endpoints |
| Evaluation of the effect of | Change from baseline in the FSHD-Composite |
| RO7204239 compared with placebo | Functional Outcome Measure total score and |
| | sub-scale scores at Week 52 of treatment |
| | Change from baseline in elbow flexion strength |
| | (right and left side) as assessed by myometry |
| | at Week 52 of treatment |
| | Change from baseline in elbow extension strength |
| | (right and left side) as assessed by myometry at |
| | Week 52 of treatment |
| | Change from baseline in shoulder abduction |
| | strength (right and left side) as assessed by |
| | myometry at Week 52 of treatment |
| | Change from baseline in knee flexion strength |
| | (right and left side) as assessed by myometry |
| | at Week 52 of treatment |
| | Change from baseline in knee extension strength |
| | (right and left side) as assessed by myometry |
| | at Week 52 of treatment |
| | Change from baseline in ankle dorsiflexion |
| | strength (right and left side) as assessed by |
| | myometry at Week 52 of treatment |
| | Change from baseline in composite MMT score at |
| | Week 52 of treatment |
| | Change in reachable work space (right and left |
| | side) at Week 52 of treatment |
| | Change from baseline in lean muscle mass as |
| | assessed by full-body DXA scan at Weeks 28 and |
| | 52 of treatment |
| | Proportion of participants with improvement in |
| | the Clinical Global Impression of Change Scale |
| | at Week 52 of treatment |
| | Proportion of participants with improvement in |
| | the Patient Global Impression of Change Scale |
| | at Week 52 of treatment |
| | Proportion of participants with improvement in |
| | the Clinical Global Impression of Severity Scale |
| | at Week 52 of treatment |
| | Proportion of participants with improvement in the |
| | Patient Global Impression of Severity Scale at |
| | Week 52 of treatment |
| Evaluation of the effect of | Change from baseline in 95th percentile of stride |
| RO7204239 compared with placebo | velocity as measured by a wearable device at Week |
| on the mobility of the participants | 52 of treatment |
| | Change from baseline in 95th percentile of stair |
| | climbing velocity at Week 52 of treatment |
| | Change from baseline in 95th percentile of stride |
| | length at Week 52 of treatment |
| | Change from baseline in wrist vertical acceleration |
| | power as measured by a wearable device at Week 52 |
| | of treatment |
| | Change from baseline in angular wrist velocity as |
| | measured by a wearable device at Week 52 of |
| | treatment |
| | Change from baseline in number of falls as measured |
| | by a wearable device at Week 52 of treatment |
| | Change from baseline in number of falls as measured |
| | by participant-reported falls at Week 52 of treatment |
| To evaluate the health-related | Change from baseline in the FSHD Health Index total |
| quality of life of participants | score and subscale scores at Week 52 of treatment |
| treated with RO7204239 compared | Change from baseline in the FSHD-Rasch-built overall |
| with placebo | disability scale total score at Week 52 of treatment |
| | Change from baseline in EuroQol EQ-5D-5L index- |
| | based score at Week 52 of treatment |
| Evaluation of RO7204239 PK/PD | Relationship between PK and PD, ADAs, safety |
| effects | and/or efficacy endpoints |

ADA = anti-drug antibody; AUC = area under the concentration -time curve; Cₘₐₓ = maximum observed concentration; C_{trough} = trough concentration; CTCAE = Common Terminology Criteria for Adverse Events; DXA = dual-energy X-ray absorptiometry; FSHD = facioscapulohumeral muscular dystrophy; MMT = manual muscle testing; MRI = magnetic resonance imaging; NCI = National Cancer Institute; PD = pharmacodynamic; PK = pharmacokinetic.

### REFERENCES

Brooks R. EuroQol: the current state of play. Health Policy 1996; 37:53-72.
Burch P M, Pogoryelova O, Palandra J, et al. Reduced serum myostatin concentrations associated with genetic muscle disease progression. J Neurol 2017; 264 (3): 541-53.
Deenan J C W, Arnts H, van der Maarel S M, et al. Population-based incidence and prevalence of facioscapulohumeral dystrophy. Neurology 2014; 83 (12): 1056-9.
Eichinger K, Heatwole C, Iyadurai S, et al. Facioscapular muscular dystrophy functional composite outcome measure. Muscle Nerve 2018; 58:72-8.
EuroQol Group. EuroQol: a new facility for the measurement of health-related quality of life. Health Policy 1990; 16:199-208.
Giesige C R, Wallace L M, Heller K N, et al. AAV-mediated follistatin gene therapy improves functional outcomes in the TIC-DUX4 mouse model of FSHD. JCI Insight 2018; 3 (22): e123538.
Hamel J, Tawil R. Facioscapulohumeral muscular dystrophy: Update on pathogenesis and future treatments. Neurotherapeutics 2018; 15 (4): 863-71.
Han J J, Kurillo G, Abresch R T, et al. Reachable workspace in facioscapulohumeral muscular dystrophy (FSHD) by Kinect. Muscle Nerve 2015; 51 (2): 168-75.
Herdman M, Gudex C, Lloyd A, et al. Development and preliminary testing of the new five-level version of EQ-5D (EQ5D-5L). Qual-Life Res 2011; 20:1727-36.
Janssen B H, Voet N B, Nabuurs C I, et al. Distinct disease phases in muscles of facioscapulohumeral dystrophy patients identified by MR detected fat infiltration. PLOS One 2014; 9 (1): e85416.
Janssen M F, Pickard A S, Golicki D, et al. Measurement properties of the EQ-5D-5L compared to the EQ-5D3L across eight patient groups: a multi-country study. Qual-Life Res 2013; 22:1717-27.
Lassche S, Voermans N C, Schreuder T, et al. Reduced specific force in patients with mild and severe facioscapulohumeral muscular dystrophy. Muscle Nerve 2021; 63:60-7.
LoRusso S, Johnson N, McDermott M P, et al. Clinical trial readiness to solve barriers to drug development in FSHD (ReSolve): Protocol of a large, international, multi-center prospective study. BMC Neurol 2019; 19:224.
Mul K, Vincenten S C C, Voermans N C, et al. Adding quantitative muscle MRI to the FSHD clinical trial toolbox. Neurology 2017; 89 (20): 2057-65.
Muramatsu, H., Kuramochi, T., Katada, H. et al. Novel myostatin-specific antibody enhances muscle strength in muscle disease models. Sci Rep 2021; 11:2160.
Personius K E, Pandya S, King W M, et al. Facioscapulohumeral dystrophy natural history study: standardization of testing procedures and reliability of measurements. The FSH DY Group. Phys Ther 1994; 74:253-63.
Preston M K, Tawil R, Wang L H. Facioscapulohumeral muscular dystrophy. In: Adam M P, Ardinger H H, Pagon R A, et al., editors. GeneReviews® [Internet]. Seattle (WA): University of Washington, Seattle, 1999 [Updated 2020 Feb. 6]: 1993-2021.
Statland J M, Tawil R. Facioscapulohumeral muscular dystrophy. Continuum (Minneap Minn) 2016; 22 (6): 1916-31.
Tasca G, Monforte M, Ottaviani P, et al. Magnetic resonance imaging in a large cohort of facioscapulohumeral muscular dystrophy patients: Pattern refinement and implications for clinical trials. Ann Neurol 2016; 79:854-64.
Tawil R, Kissel J T, Heatwole C, et al. Evidence-based guideline summary: Evaluation, diagnosis, and management of facioscapulohumeral muscular dystrophy: Report of the Guideline Development, Dissemination, and Implementation Subcommittee of the American Academy of Neurology and the Practice Issues Review Panel of the American Association of Neuromuscular & Electrodiagnostic Medicine. Neurology 2015; 85(4):357-64.
Wagner K R. Facioscapulohumeral Muscular Dystrophies. Continuum (Minneap Minn) 2019; 25(6):1662-81.

1. An anti-myostatin antibody for use in the treatment, prevention, delaying progression and/or amelioration of Facioscapulohumeral muscular dystrophy (FSHD).

2. The anti-myostatin antibody for the use of claim 1, wherein the antimyostatin antibody binds to latent myostatin and does not bind to mature myostatin, wherein the antibody blocks the non-proteolytic, spontaneous release of mature myostatin from latent myostatin and inhibits activation of myostatin.

3. The anti-myostatin antibody for the use of claim 1, wherein the anti-myostatin anti-body comprises the six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

4. The anti-myostatin antibody for the use of claim 1, wherein the anti-myostatin anti-body comprises a VH chain comprising the amino acid sequence of SEQ ID 7 and a VL chain comprising the amino acid sequence of SEQ ID 8.

5. The anti-myostatin antibody for the use of claim 1, wherein the anti-myostatin anti-body comprises a heavy chain comprising the amino acid sequence of SEQ ID 9 and a light chain comprising the amino acid sequence of SEQ ID 10.

6. The anti-myostatin antibody for the use of claim 1, in patient (in particular a patient in need thereof), particularly wherein the patient is a human (such as a male or female human).

7. The anti-myostatin antibody for the use of claim 1, wherein the anti-myostatin anti-body is administered every four weeks in a dose of 90 mg to a subject, preferably a human subject.

8. The anti-myostatin antibody for the use of claim 1, wherein the anti-myostatin anti-body is administered subcutaneously.
